(19) United States
(12) Patent Application Publication
(10) Pub. No.: US 2011/0082191 A1
(43) Pub. Date: Apr. 7, 2011

(54) METHOD FOR TREATING OR PREVENTING RADIATION DAMAGE BY IN VIVO GENE THERAPY

(75) Inventors: Weiwen Deng, Grand Rapids, MI (US); Aly S. Abdel-Mageed, Grand Rapids, MI (US)

(73) Assignee: SPECTRUM HEALTH INNOVATIONS, LLC, Grand Rapids, MI (US)

(21) Appl. No.: 12/999,403

(22) PCT Filed: Jun. 26, 2009
(86) PCT No.: PCT/US09/48760

§ 371 Date: Dec. 16, 2010

Related U.S. Application Data
(60) Provisional application No. 61/079,897, filed on Jul. 11, 2008.
(60) Provisional application No. 61/075,763, filed on Jun. 26, 2008.

(51) Int. Cl. A61K 48/00; A61K 31/7088

(52) U.S. Cl. 514/44 R

(57) ABSTRACT

The current embodiment provides a method and gene therapy for treating or preventing radiation damage using a vector carrying a gene for extracellular superoxide dismutase (ECSOD). The gene therapy can be used to treat a patient with an effective amount of the therapy to limit damage resulting from exposure to radiation and radiation-like damage.

### BACKGROUND OF THE INVENTION

The present invention relates to radiation damage, and more particularly, to treatments of radiation damage.

The risk to civilians, police, and military personnel of being exposed to lethal doses of ionizing radiation is greater now than ever before due to a growing possibility of a nuclear terrorism event (Zenk, Expert Opin Investig Drugs. 16: 767-70 [2007]). A typical scenario assumes that suicide terrorists would bring a ¹³⁷Cs source into a major subway station of a metropolitan city, unshield the source, and attempt to expose as many people as possible to lethal γ radiation. As a result, several thousand or more people could be irradiated to varying dosages of whole body radiation and all would be at risk for developing some degree of acute radiation syndrome (ARS) (Fliedner, Curr Opin Hematol. 13: 436-44 [2006]). A significant number of the victims are believed to die of ARS within 60 days after receiving 4-6 Gy whole body exposure (sooner with higher doses). Clinical observation has confirmed that peripheral blood lymphocyte count drops rapidly in a dose-dependent manner within 1-2 days after total body irradiation (TBI) exposure (Fliedner, Curr Opin Hematol. 13: 436-44 [2006]; Greenberger, Pharmacogenomics 7: 1141-5 [2006]).

ARS is a condition caused by a brief whole body exposure to more than one sievert (Sv) dose equivalent of radiation. ARS is initially characterized by anorexia, nausea, and vomiting but can progress to hematological, gastrointestinal, neurological, pulmonary, and other major organ dysfunction. The degree of symptom severity of ARS is directly correlated to the absorbed dose of radiation. The cells of the body that are most vulnerable to damage from radiation are the rapidly dividing cells of bone marrow and intestinal lining (Mettler and Voelz, N Engl J Med. 346: 1554-61 [2002]; Zenk, Expert Opin Investig Drugs. 16: 767-70 [2007]). At present, no approved drugs or therapies are available for the prevention or treatment of ARS, despite the critical nature of this national security threat (Weisdorf et al., Biol Blood Marrow Transplant 12: 672-82 [2006]; Zenk, Expert Opin Investig Drugs. 16: 767-70 [2007]; Chao, Exp Hematol. 35(4 Suppl 1): 24-7 [2007]; Greenberger, Gene Ther. 15: 100-8 [2008]).

Radiation exposure also produces delayed radiation effects such as lifespan shortening, cataractogenesis, and carcinogenesis, which can occur months to decades later (Epperly et al., Radiat Res. 170: 437-43 [2008]). Further, cataract of the eyes and cancer development are the known late-effects of exposure to space radiation in astronauts, military aviators, and flight crews (Parker, Sci. Am. 294: 40-7 [2006]).

The formation of reactive oxygen species (ROS) such as superoxide anion (O₂⁻) following irradiation is a major determinant of lethality following fatal whole body radiation exposure (Zwacka et al., Hum Gene Ther. 9: 1381-6 [1998]; Mitchell et al., Ann N Y Acad Sci. 899: 28-43 [2000]; Murray and McEwan, Cancer Biotherapy & Radiopharmaceuticals 22: 1-23 [2007]). Radiation exposure induces oxidative damage to bone marrow and gastrointestinal tract which is the major factor causing bone marrow failure and gastrointestinal damage (Greenberger, Pharmacogenomics 7: 1141-5 [2006]; Zenk, Expert Opin Investig Drugs. 16: 767-70 [2007]). Radiation dosage is expressed in gray (Gy). At a dose of <1 Gy, the damage to cells is not severe and almost all victims survive. At a dose of 1-8 Gy, there is damage to bone marrow stem cells, resulting in hematopoietic dysfunction manifesting as decreased numbers of white blood cells and platelets, which lead to an increased susceptibility to infection and bleeding. At a dose of 8-30 Gy, there is serious damage to the gastrointestinal tract. The absorbed dose of radiation at which 50% of exposed individuals will die without medical support is estimated to be 3.25 Gy (Waselenko et al., Ann Intern Med. 140: 1037-52 [2004]; Zenk, Expert Opin Investig Drugs. 16: 767-70 [2007]). It is observed that irradiated tissues and organs release ROS for days to months after ionizing radiation exposure (Greenberger and Epperly, In: Progress in Gene Therapy. Columbus Frank (Ed.), Nova Science Publications, NY, USA, p110-8 [2005]).

A radioprotective agent functions to protect critical body tissues against low to moderate doses of ionizing radiation and the in situ generated free radicals associated with biological tissues being exposed to such radiation. Radioprotective agents are beneficially administered to patients receiving radioisotope and radiation treatments, as well as to protect individuals entering radiation-contaminated environments. Such radioprotective agents serve antimutagenic and anticarcinogenic roles within tissues containing such agents.

The development of radioprotective agents has been the subject of intense research in view of their potential use in a radiation environment, such as space exploration, radiotherapy, and even nuclear war, for many decades. However, no ideal, safe synthetic radioprotectors are available to date, so the search for alternative sources, including plants, continues. Presently available methods and compositions for treating radiation damage require the administration of high doses of agents such as pharmaceuticals or other chemical additives by parenteral routes within a short time frame before or after the radiation or chemical insult (See e.g., Bump and Malaker (eds.), Radioprotectors: Chemical, Biological, and Clinical Perspectives, CRC Press, Washington, D.C. [1997]). Therefore, this precludes their use as a long-term prophylactic measure for use in protection against unanticipated radiation injury.

Additionally, most radioprotective agents only have a short duration of action. Many active agents lose viability over time and may not exhibit good bulking activity or good film forming characteristics. Many active agents are insoluble in water, and thus the active agents have to be applied as aqueous emulsions. For instance, proteins and peptides may be desirable active agents, particularly for protein-based applications, but incorporation into formulations may be problematic due to their generally high levels of hydrophobicity, and incorporation into material substrates may subject them to laundering or other cleaning effects, causing loss of the active agent as well as functional efficacy, over time. This limits the potential feasibility of using such agents.

More specifically, radioprotective agents reduce the biological effects of radiation. They may be administrated before and/or after radiation exposure and can protect the organism from radiation-induced lethality. Radioprotectors have been shown to operate by a variety of different mechanisms (for review, see e.g., Bump and Malaker (eds.), Radioprotectors: Chemical, Biological, and Clinical Perspectives, CRC Press, Washington, D.C. [1997]). The mechanisms of protection can be based on the radioprotector's antioxidant properties (Weiss and Landauer, Ann. NY Acad. Sci., 899:44-60 [2000]), estrogenic activity (Miernicki et al., Soc. Neurosci. Abstr., 16:1054 [1990]; and Patt et al., Amer. J. Physiol., 159:269-280 [1949]), and/or in some cases, the ability to inhibit protein kinase(s) involved in signal transduction (Liu et al., Oncogene, 19: 571-579[2000]).

Ionizing radiation increases formation of O₂⁻ which causes DNA strand breaks, oxidizes membrane lipids, and reacts with nitric oxide to form the toxic peroxonitrite. All of these oxidative damage processes can lead to cell apoptosis (Kanai et al., Am J Physiol Renal Physiol. 283: F1304-12 [2002]; Jung et al, Circ Res. 93: 622-9 [2003]; Greenberger and Epperly, In: Progress in Gene Therapy. Columbus Frank (Ed.), Nova Science Publications, NY, USA, p110-8 [2005]; Rodemann and Blaese, Semin Radiat Oncol. 17: 81-8 [2007]). Therefore, strategies and compositions to decrease O₂⁻ have been searched for in order to alleviate radiation-induced oxidative damage.

For example, superoxide dismutase (SOD), an antioxidant enzyme catalyzing the dismutation of O₂⁻, alleviates oxidative damage. Three SOD isoforms have been identified: cytosolic copper-zinc SOD (CuZnSOD), mitochondrial manganese SOD (MnSOD), and extracellular SOD (ECSOD). ECSOD gene therapy has recently been shown to be effective for a variety of diseases involving oxidative damage (Bivalacqua et al., Am J Physiol Heart Circ Physiol. 284: H1408-21 [2003]; Bivalacqua et al., J Sex Med. 2: 187-97 [2005]; Brown et al., Am J Physiol Heart Circ Physiol. 290: H2600-5 [2006]; Heistad, Arterioscler Thromb Vasc Biol. 26: 689-95 [2006]), but has not been used to treating radiation damage.

Since the function of MnSOD is to remove O₂⁻ produced in mitochondria, augmentation of MnSOD expression before irradiation is thought to inhibit radiation-induced cell apoptosis. It has been shown that gene transfer of MnSOD protects against radiation-induced tissue damage in mice when administrated prior to irradiation. The mechanism was considered to be a stabilization of mitochondrial membrane through the removal of O₂⁻ produced in mitochondria by MnSOD (Fridovich, Annu Rev Biochem. 64: 97-112 [1995]; Kanai et al., Am J Physiol Renal Physiol. 283: F1304-12 [2002]; Epperly et al., Radiat Res. 157: 568-77 [2002]; Epperly et al., Radiat Res. 160: 568-78 [2003]; Heistad, Arterioscler Thromb Vasc Biol. 26: 689-95 [2006]). MnSOD gene therapy studies show that overexpression of MnSOD prior to radiation exposure can provide radioprotection to normal tissues in irradiated animals (Epperly et al., Int J Radiat Oncol Biol Phys. 43: 169-81 [1999]; Kanai et al., Am J Physiol Renal Physiol. 283: F1304-12 [2002]; Epperly et al., Mil Med. 167(2 Suppl): 71-3 [2002]; Niu et al., In Vivo. 19: 965-74 [2005]). In one study, intratracheal injection of adenovirus containing human MnSOD for the overexpression of MnSOD in the lungs of mice prior to irradiation prevents mice from irradiation-induced organizing alveolitis (Epperly et al., Int J Radiat Oncol Biol Phys. 43: 169-81 [1999]). In another study, MnSOD plasmid was administrated to mice 24 hours prior to esophageal irradiation and it protected esophageal progenitors of squamous epithelium (Niu et al., In Vivo. 19: 965-74 [2005]). However, MnSOD radioprotective gene therapy has no therapeutic effect when MnSOD gene construct is administrated after radiation exposure (Greenberger et al., Curr Gene Ther. 3: 183-95 [2003]; Greenberger, Pharmacogenomics 7: 1141-5 [2006]; Greenberger, Gene Ther. 15: 100-8 [2008]).

ECSOD is a secretory Cu and Zn-containing tetrameric glycoprotein. ECSOD is the only SOD isoform that is released from cells. ECSOD is produced and secreted only by macrophages, smooth muscle cells, fibroblasts, and glia cells. It exists primarily in the interstitial space of tissues, plasma, and lymph (Marklund, Biochem J. 266: 213-9 [1990]; Stralin and Marklund, Biochem J. 298 (Pt 2): 347-52 [1994]; Choung et al., Exp Dermatol. 13: 691-9 [2004]). ECSOD is suggested to be a major determinant of nitric oxide bioavailability for the maintenance of vascular function (Jung et al., Circ Res. 93: 622-9 [2003]; Faraci and Didion, Arterioscler Thromb Vasc Biol. 24: 1367-73 [2004]). As ECSOD is found in the extracellular matrix of tissues, it is ideally situated to prevent cell and tissue damage initiated by extracellularly produced O₂⁻ (Fattman et al., Free Radic Biol Med. 35: 236-56 [2003]). Animal studies have demonstrated that adenovirus mediated ECSOD gene therapy is effective in treating a variety of cardiovascular diseases (Fennell et al., Gene Ther. 9: 110-7 [2002]; Chu, Methods Mol Med. 108: 351-61 [2005]; Bivalacqua et al., J Sex Med. 2: 187-97 [2005]; Brown et al., Am J Physiol Heart Circ Physiol 290: H2600-5 [2006]; Heistad, Arterioscler Thromb Vasc Biol. 26: 689-95 [2006]). It is also demonstrated that overexpression of ECSOD reduces acute radiation induced lung toxicity (Kang et al., Int J Radiat Oncol Biol Phys. 57: 1056-66 [2003]; Rabbani et al., BMC Cancer. 5: 1-13 [2005]). However, no effective therapy has been created to date containing ECSOD.

Despite all of the above information, there remains no good treatment of radiation damage and no animal model to study the effects both of radiation damage and the proposed treatments in more detail.

### SUMMARY OF THE INVENTION

A method and therapeutic is provided for at least one of treating and/or preventing radiation damage by administering to a patient in need of treatment at least one therapeutically effective amount of a vector carrying an extracellular superoxide dismutase gene to produce and secrete exogenous extracellular superoxide dismutase.

In one embodiment, the vector is administered in a medically acceptable manner. Examples of such acceptable manners include, but are not limited to, intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and combinations thereof.

In yet another embodiment, the vector can be administered to a patient at a variety of locations including, but not limited to, systemically, at the site of injury, at an adjacent site to the site of injury, and at a site remote from the site of injury, wherein the mesenchymal stem cells migrate to the site of injury after administration.

In another embodiment, the vectors are administered in multiple therapeutically effective amounts. The repeated administration provides additional production of ECSOD at the location of treatment.

Additionally, the vector can be administered in combination with another therapeutic. The additional therapeutic can be selected based on the desired end result. Examples of such therapeutics include, but are not limited to, radiotherapeutics, chemotherapeutics, and immunomodulators.

In another further embodiment, a therapeutic for treating and/or preventing radiation related damage is provided. The therapeutic is formed of a vector carrying an extracellular superoxide dismutase gene to produce and secrete exogenous extracellular superoxide dismutase. The therapeutic can be used in the method described above for treating and preventing radiation damage.

The vectors for use in the therapeutic can be any medically acceptable vector. Examples of such medically acceptable vectors include, but are not limited to, adenovirus vectors, retrovirus vectors, adeno-associated virus vectors, herpes simplex virus vectors, SV 40 vectors, polyoma virus vectors, papilloma virus vectors, picarnovirus vectors, vaccinia virus vectors, a helper-dependent adenovirus, a gutless adenovirus, and plasmid.

In yet a further embodiment, the extracellular superoxide dismutase gene is a human extracellular superoxide dismutase gene or a murine extracellular superoxide dismutase gene.

The therapeutic can be used to treat or prevent radiation damage. Examples of radiation damage include, but are not limited to, cell injury, tissue damage, organ dysfunction, acute radiation syndrome, and delayed radiation effects such as radiation-induced lifespan shortening, cataractogenesis, and carcinogenesis.

In another embodiment the therapeutic can be combined with another unrelated therapeutic, or used in combination with other treatments or therapeutics. Examples of unrelated therapeutics include, but are not limited to, radiotherapeutics and chemotherapeutics.

These and other objects, advantages, and features of the invention will be more fully understood and appreciated by reference to the description of the current embodiment and the drawings.

### BRIEF DESCRIPTION OF THE DRAWINGS

FIGS. 1A and 1B show that radiation damage is dose dependent. FIG. 1A is a graph depicting the effect of an irradiation dose on mouse body weight loss. FIG. 1B is a Kaplan-Meier survival curve depicting the effect of an irradiation dose on mouse survival.

FIGS. 2A and 2B show that in vivo extracellular superoxide dismutase (ECSOD) gene therapy has a radioprotective effect in irradiated mice. FIG. 2A is a graph depicting the effect of intravenous adenoviral-mediated gene transfer of ECSOD on mouse body weight loss in irradiated mice. FIG. 2B is a Kaplan-Meier survival curve demonstrating that intravenous treatment with adenovirus carrying ECSOD gene improves survival of irradiated mice.

FIGS. 3A and 3B show the efficacy of adenoviral gene transfer in mouse mesenchymal stem cells (mMSCs). FIG. 3A is a graph depicting the secretion of biologically active extracellular superoxide dismutase (ECSOD) by Ad5CMVECSOD-transduced mMSCs. FIG. 3B is a photomicrograph depicting the expression of nuclear targeted β-galactosidase by Ad5CMVntlacZ-transduced mMSCs.

FIGS. 4A and 4B show the efficacy of adenoviral gene transfer in human mesenchymal stem cells (hMSCs). FIG. 4A is a graph depicting the secretion of biologically active extracellular superoxide dismutase (ECSOD) by Ad5CMVECSOD-transduced hMSCs. FIG. 4B is a photomicrograph depicting the expression of nuclear targeted β-galactosidase by Ad5CMVntlacZ-transduced hMSCs.

### DETAILED DESCRIPTION OF THE CURRENT EMBODIMENT

In the current embodiment, a gene therapy method and therapeutic for treating radiation damage using in vivo delivery of a gene therapy containing extracellular superoxide dismutase (ECSOD) are provided. Also provided is a method of administering a gene therapy carrying human ECSOD gene to produce and secrete exogenous ECSOD. Further provided is a method of ECSOD gene therapy for improving survival after whole-body radiation exposure. The gene therapy method and therapeutic provide a human patient with an effective medical countermeasure to radiation exposure to be expected as a consequence of a number of problems including, but not limited to, a radiation accident, nuclear accident, nuclear terrorism, nuclear war, other radiological emergencies, space travel, radiation therapy, and diagnostic radiology. The gene therapy method and therapeutic can also be used to pre-treat and/or treat other forms of cellular damage that have mechanisms of action similar to radiation. The gene therapy is a vector containing extracellular superoxide dismutase (ECSOD).

The term “gene therapy” as used herein is intended to include any therapy capable of providing ECSOD to a patient. The form of the gene therapy can be any therapy known by those of skill in the art to be capable of administering ECSOD to a patient. For example, vectors are known to be used for such purposes.

In another embodiment, the “gene therapy” can include any therapy capable of providing ECSOD protein to a patient. The form of the therapy can be any therapy known by those of skill in the art to be capable of administering ECSOD protein to a patient. Additionally, the protein can be used in conjunction with mesenchymal stem cells (MSC) to provide an alternative treatment.

The term “vector” as used herein is intended to refer to a vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides, including a nutrient-regulatable expression control element in operable linkage with a nucleic acid, and expressing the transcribed antisense or encoded protein in cells in vitro or in vivo. Examples of such vectors include, but are not limited to (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picarnovirus vectors; (i) vaccinia virus vectors; (j) a helper-dependent or gutless adenovirus; and (k) a plasmid.

The vector is therefore capable of transferring gene sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

A transfer vector is a DNA molecule which contains, inter alia, genetic information that insures its own replication when transferred to a host microorganism strain. Examples of transfer vectors commonly used in bacterial genetics are plasmids and the DNA of certain bacteriophages.

“Plasmid” is the term applied to any autonomously replicating DNA unit which might be found in a microbial cell, other than the genome of the host cell itself. A plasmid is not genetically linked to the chromosome of the host cell. Plasmid DNA's exist as double stranded ring structures generally on the order of a few million daltons molecular weight, although some are greater than 10⁸ daltons in molecular weight. They usually represent only a small percent of the total DNA of the cell.

Transfer vector DNA is usually separable from host cell DNA by virtue of the great difference in size between them. Transfer vectors carry genetic information enabling them to replicate within the host cell, in some cases independently of the rate of host cell division. Some plasmids have the property that their replication rate can be controlled by the investigator by variations in the growth conditions.

Plasmid DNA exists as a closed ring. However, by appropriate techniques, the ring may be opened, a fragment of heterologous DNA inserted, and the ring reclosed, forming an enlarged molecule comprising the inserted DNA segment. Bacteriophage DNA may carry a segment of heterologous DNA inserted in place of certain nonessential phage genes. Either way, the transfer vector serves as a carrier or vector for an inserted fragment of heterologous DNA.

Transfer is accomplished by a process known as transformation. During transformation, bacterial cells mixed with plasmid DNA incorporate entire plasmid molecules into the cells. It is possible to maximize the proportion of bacterial cells capable of taking up plasmid DNA and hence of being transformed, by certain empirically determined treatments. Once a cell has incorporated a plasmid, the latter is replicated within the cell and the plasmid replicas are distributed to the daughter cells when the cell divides. Any genetic information contained in the nucleotide sequence of the plasmid DNA can, in principle, be expressed in the host cell.

Typically, a transformed host cell is recognized by its acquisition of traits carried on the plasmid, such as resistance to certain antibiotics. Different plasmids are recognizable by the different capabilities or combination of capabilities which they confer upon the host cell containing them. Any given plasmid may be made in quantity by growing a pure culture of cells containing the plasmid and isolating the plasmid DNA therefrom.

“Adenoviruses (Ad)” are double-stranded linear DNA viruses with a 36 kb genome. Several features of adenovirus have made them useful as transgene delivery vehicles for therapeutic applications, such as facilitating in vivo gene delivery.

“Lentiviral vector and recombinant lentiviral vector” refer to a nucleic acid construct which carries, and within certain embodiments, is capable of directing the expression of a nucleic acid molecule of interest. The lentiviral vector include at least one transcriptional promoter/enhancer or locus defining element(s), or other elements which control gene expression by other means such as alternate splicing, nuclear RNA export, post-translational modification of messenger, or post-transcriptional modification of protein. Such vector constructs can also include a packaging signal, long terminal repeats (LTRS) or portion thereof, and positive and negative strand primer binding sites appropriate to the retrovirus used (if these are not already present in the retroviral vector). The recombinant lentivirus is capable of reverse transcribing its genetic material (RNA) into DNA and incorporating this genetic material into a host cell's DNA upon infection. Lentiviral vector particles may have a lentiviral envelope, a non-lentiviral envelope (e.g., an ampho or VSV-G envelope), or a chimeric envelope.

A vector generally contains at least an origin of replication for propagation in a cell. Control elements, including expression control elements (e.g., nutrient-regulatable) as set forth herein, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

“Nucleic acid expression vector” or “expression cassette” refers to an assembly which is capable of directing the expression of a sequence or gene of interest. The nucleic acid expression vector includes a promoter which is operably linked to the sequences or gene(s) of interest. Other control elements may be present as well. In addition to the components of the expression cassette, the plasmid construct may also include a bacterial origin of replication, one or more selectable markers, a signal which allows the plasmid construct to exist as single-stranded DNA (e.g., a M13 origin of replication), a multiple cloning site, and a “mammalian” origin of replication (e.g., a SV40 or adenovirus origin of replication).

Vectors can include a selection marker. As is known in the art, “selection marker” or equivalents means genes that allow the selection of cells containing the gene. “Positive selection” refers to a process whereby only the cells that contain the positive selection marker will survive upon exposure to the positive selection agent or be marked. For example, drug resistance is a common positive selection marker; cells containing the positive selection marker will survive in culture medium containing the selection drug, and those which do not contain the resistance gene will die.

“Expression control elements” include polynucleotides, such as promoters and enhancers that influence expression of an operably linked nucleic acid. Expression control elements and promoters include those active in a particular tissue or cell type, referred to herein as a “tissue-specific expression control elements/promoters.” Tissue-specific expression control elements are typically active in specific cells or tissues because they are recognized by transcriptional activator proteins, or other regulators of transcription, that are unique to a specific cell or tissue type.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences. For example, the sequences and/or vectors described herein may also include one or more additional sequences that may optimize translation and/or termination including, but not limited to, a Kozak sequence (e.g., GCCACC placed in front (5′) of the ATG of the codon-optimized wild-type leader or any other suitable leader sequence (e.g., tpa1, tpa2, wtLnat (native wild-type leader)) or a termination sequence (e.g., TAA or TAAA placed after (3′) the coding sequence

The term “radiation damage” as used herein is intended to include, but is not limited to, cell injury, tissue damage, tissue dysfunction, acute radiation syndrome, delayed radiation effects such as radiation-induced lifespan shortening, cataractogenesis, and carcinogenesis, and other like damage relating to or caused from exposure to radiation, as well as damage caused by other substances but which has a similar effect on cells and tissues as radiation damage. The radiation exposure can be a consequence of a number of problems including, but not limited to a radiation accident, nuclear accident, nuclear terrorism, nuclear war, other radiological emergencies, space travel, diagnostic radiology, as well as radiation therapies, chemotherapeutics, and radio-sensitizers. Space travel can include, but not limited to, exposure to space radiation in astronauts, military aviators, and flight crews. Radiation-induced cataract and cancer become a major health risk in long-duration space flights such as missions to the International Space Station, the moon and Mars. Diagnostic radiology can include, but not limited to, X-ray radiographing, CT scanning, and nuclear medicine imaging. Radiation therapy can include a radiation treatment of cancers including, but not limited to, leukemia, lymphoma, brain tumor, thyroid tumor, lung cancer, liver cancer, breast cancer, cervical cancer, ovarian cancer, prostate cancer, endometrial cancer, bladder cancer, colorectal cancer, and other similar cancers or diseases.

The term “ECSOD” as used herein is intended to include, but is not limited to, ECSOD of recombinant origin. This form is easily available in large quantities, but ECSOD is also available from other sources. Thus, the ECSOD can also be of cell line origin, i.e. derived from a cell line producing the protein in significant quantities, such as a cell line derived from blood or lung, blood vessel, pancreas, uterus, prostate gland, placenta or umbilical cord tissue, and neoplastic tissue. Endothelial cells or fibroblasts can also be sources of ECSOD. ECSOD is an antioxidant enzyme catalyzing the dismutation of superoxide anion. The ECSOD can be human extracellular superoxide dismutase or a mammalian extracellular superoxide dismutase.

The ECSOD can also be derived from tissue found to be relatively rich in ECSOD. Accordingly, the current embodiment further relates to ECSOD of placenta or umbilical cord origin as these tissues have been formed to contain reasonably large amounts of ECSOD compared to other types of tissue, and are also more easily available than, for instance, lung, uterus or pancreas tissue. Although these tissues contain relatively larger amounts of ECSOD, these amounts are far smaller than those obtainable by recombinant DNA techniques, and therefore, placenta or umbilical cord ECSOD is particularly indicated for special purposes requiring only minor amounts of ECSOD.

As used herein, the term “patient” or “subject” refers to a warm-blooded animal such as a mouse, rat, cat, dog, cow, horse, monkey, and human.

The term “mesenchymal stem cells (“MSCs”)” as used herein is intended to include, but is not limited to, multipotent cells that can differentiate into a variety of cell types. Mesenchymal stem cells (MSCs), also known as marrow stromal cells, are a subset of adult stem cells from bone marrow. The cells have multilineage differentiation potential and contribute to the regeneration of mesenchymal tissues such as bone, cartilage, fat, and muscle (Prockop, Science 276: 71-4 [1997]; Ferrari et al., Science 279: 1528-30 [1998]; Pittenger et al., Science 284: 143-7 [1999]; Dominici et al., Cytotherapy 8: 315-7 [2006]). Since MSCs are relatively easy to isolate and expand ex vivo, the cells have been used for tissue repair or regeneration in adult stem cell-based cell and gene therapy of a variety of diseases including osteogenesis imperfects, stroke, myocardiac infarction, pulmonary hypertension, and erectile dysfunction (Horwitz et al., Nat Med. 5: 309-13 [1999]; Zhang et al., Circ Res. 90: 284-8 [2002]; Toma et al., Circulation 105: 93-8 [2002]; Baber et al., Am J Physiol Heart Circ Physiol 292: H1120-8 [2007]; Deng et al., Am J Physiol Cell Physiol 285: C1322-29 [2003]; Deng et al., Stem Cells 22: 1279-91 [2004]; Deng et al., Int J Impot Res. 17 suppl 1: S57-63 [2005]; Deng et al., Life Sci. 78: 1830-8 [2006]; Bivalacqua et al., Am J Physiol Heart Circ Physiol. 292: H1278-90 [2007]). For example, MSCs have been shown to differentiate into osteoblasts, chondrocytes, myocytes, adipocytes, and beta-pancreatic islets cells. MSCs have a large capacity for self-renewal while maintaining their multipotency. MSCs can be isolated from bone marrow, umbilical cord blood, adipose tissue, and peripheral blood. Also, mesenchymal stem cells can be autologous, allogeneic, syngeneic, and xenogeneic mesenchymal stem cells, with respect to the individual or mammalian subject that is receiving the MSC treatment. The MSCs can be derived from human or other mammalian stem cells. The MSCs are genetically modified with extracellular superoxide dismutase using a vector to transfer the cDNA of extracellular superoxide dismutase into the MSCs for the production and secretion of extracellular superoxide dismutase by the MSCs.

A “protective amount of the gene therapy” as used herein refers to that amount which is both non-toxic and creates the desired effect, wherein the desired effect is eliminating or reducing in severity or in extent the deleterious cellular effects caused by exposure to or treatment with radiation or radiation-like compositions. The gene therapy can be administered after irradiation in an amount that is effective for diminishing damage to the respiratory, gastrointestinal, hematopoietic, or other systems after sublethal irradiation or for increasing the survival rate after lethal irradiation. The gene therapy is also effective when administered prior to or during exposure to radiation.

The gene therapy can be administered, either before and/or after exposure to radiation, in a single or multiple dose administration to a subject or patient. The gene therapy of the current embodiment can also be administered in conjunction with other therapeutic agents. A protective amount of the gene therapy also refers to that amount which is effective, upon single or multiple dose administration to humans and other living organisms, in eliminating or reducing in severity or in extent the destructive cellular effects caused by exposure to ionizing radiation.

A protective amount of the gene therapy can be administered to a subject or a patient using techniques known to those of skill in the art and by observing results obtained under analogous circumstances. The protective amount of the gene therapy can be readily determined by one of ordinary skill in the art. In determining the protective amount or dose, a number of factors are considered by one skilled in the art, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances all of which are well known to those of skill in the art. A protective amount of the gene therapy for administration to a mammal or patient will vary depending upon the amount of radiation exposure and the time period of radiation exposure, with the upper limit of the composition limited by the toxicity of a large dose. A larger dose of the gene therapy will be required for lethal radiation exposure, while a lower dose can be used where the radiation exposure is sub-lethal or chronic.

The details of the dosing schedule for the gene therapy of the current embodiment necessary to provide the maximum selective protective effect upon exposure to ionizing radiation can be readily determined by one skilled in the art by the use of known techniques and by observing results obtained under analogous circumstances.

The current embodiment provides a method for treating radiation damage in a mammalian subject or a human individual. The method can entail in vivo administration of a gene therapy, which is a vector carrying an ECSOD gene to produce and secrete exogenous ECSOD with the subject. The vectors are capable of transferring the secreting extracellular superoxide dismutase to neutralize or eliminate the toxic superoxide anion that is elicited by ionizing radiation. The gene therapy is administered in a therapeutically acceptable amount to a subject in need of treatment. The method is particularly useful for the treatment of radiation damage after radiation exposure as a consequence of radiation accident, nuclear accident, nuclear terrorist attack, nuclear war, other radiological emergencies, space travel, radiation therapy, and diagnostic radiology. In one embodiment, the treatment is provided to pregnant women either before or after an X-ray to prevent or treat damage resulting from the radiation exposure.

In another embodiment, the gene therapy is used in vitro for creating a cellular culture or growth capable of secreting exogenous ECSOD. The method entails in vitro administration of a gene therapy, which is a vector carrying an ECSOD gene to produce and secrete exogenous ECSOD, to cell contained in a culture medium. Besides MSCs, other types of cells may also be used as vehicles to produce and secrete exogenous ECSOD.

In another embodiment, the method is used for the prevention and prophylactic treatment of radiation damage before radiation exposure as a consequence of radiation accident, nuclear accident, nuclear terrorist attack, nuclear war, other radiological emergencies, space travel, radiation therapy, and diagnostic radiology. For example, subjects that are likely to enter into a location susceptible to such attacks can receive a preventative treatment. This will either prevent or at least limit the damage caused by the exposure to radiation.

In another embodiment the method is further useful for the treatment of radiation damage to normal tissues after radiation therapy in tumor patients. The gene therapy can be used to treat radiation damage to normal tissues after or during radiation therapy in tumor patients. The gene therapy can decrease the severity of damage to normal tissues, particularly bone marrow, spleen, gastrointestinal tract, and healthy tissues near the tumor, caused by radiation therapy. The method is also useful for the prevention and prophylactic treatment of radiation damage to normal tissues before radiation therapy in tumor patients.

The current embodiment provides a method that protects cells and living organisms from deleterious cellular effects related either to exposure to radiation or exposure to other substances that cause damage similar to radiation. The treatment functions by at least one of preventing and/or eliminating the harmful effects or by reducing their severity. The subject to be protected can be administered the gene therapy of the current embodiment prior to, during, or after exposure of the cell to radiation. In another embodiment, the gene therapy of the current embodiment can provide a protective effect in the cell and the subject by eliminating or reducing the severity of the detrimental cellular effects that would otherwise be caused by the exposure. Therefore, the gene therapy of the current embodiment enables survival or lengthens survival of living organisms in otherwise lethal conditions.

More particularly, in another embodiment provides a method of protecting non-cancer, or normal, cells of a subject from deleterious cellular effects caused by exposure of the mammal to ionizing radiation. The gene therapy provides a protection of normal cells during exposure to radiation, such as during radiation therapy or diagnostic procedures such as x-rays and CAT scans. The cancer cells, if protected at all, are protected to a lesser extent than normal cells. The current embodiment provides a method whereby the deleterious cellular effects on non-cancer cells caused by exposure of the mammal to radiation are eliminated or reduced in severity or in extent. This treatment enables greater amounts of radiation to be administered to a patient without the detrimental side effects. The gene therapy may also protect the cells against the deleterious effects of conventional doses or accidental exposure to high doses of chemotherapeutic agents.

Additionally, the gene therapy can be administered in a higher dose to provide a systemic protective effect. One benefit of the systemic effect is that a dose of gene therapy can be administered to a patient and provide the desired effect at a variety of locations. This alleviates the need to locate all locations in need of treatment. The gene therapy can be administered via intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and the like. Administration of these compositions can be via any common route so long as the target tissue is available via that route. The routes of administration will vary with the location and nature of damage. One skilled in the art of preparing formulations can readily select the proper form and mode of administration depending upon the particular characteristics of the compound selected the disease state to be treated, the stage of the disease, and other relevant circumstances. The gene therapy can be administrated to the human individual or mammalian subject systemically, at the site of injury, at an adjacent site to the site of injury, and where following administrating the cells migrate to the site of injury. The details of the dosing schedule for the gene therapy are the amount necessary to provide the maximum selective protective effect upon exposure to ionizing radiation, which can be readily determined by one skilled in the art by the use of known techniques and by observing results obtained under analogous circumstances.

In a further embodiment, a replicable expression vector is used that includes a DNA sequence encoding ECSOD. In the present context, the term “replicable” means that the vector is able to replicate in a given type of host cell into which it has been introduced. The vector may be one carrying any DNA sequence encoding for ECSOD or any suitable modification thereof as explained above. Immediately upstream of this sequence (the coding sequence of ECSOD) there may be provided a sequence coding for a signal peptide, the presence of which ensures secretion of the ECSOD expressed by host cells harboring the vector. It should be noted that this signal sequence (and the signal peptide encoded by it) in itself forms an aspect of the current embodiment, and it is contemplated that it may be inserted upstream of DNA sequences coding for other proteins or peptides so as to obtain secretion of the resulting products from the gene therapy.

The vector may be any vector that may conveniently be subjected to recombinant DNA procedures, and the choice of vector will often depend on the host cell into which it is to be introduced. Thus, the vector may be an autonomously replicating vector, i.e. a vector that exists as an extrachromosomal entity, the replication of which is independent of chromosomal replication; examples of such a vector are a plasmid, phage, cosmid, mini-chromosome or virus. Alternatively, the vector may be one which, when introduced in a host cell, is integrated in the host cell genome and replicated together with the chromosome(s) into which it has been integrated.

Additionally, the gene therapy can be administered in a higher dose to provide a systemic protective effect. The benefit of the systemic effect is that a dose of gene therapy can be administered to a patient and provide the desired effect at any necessary locations. This alleviates the need to locate all locations in need of treatment.

The gene therapy of the invention can be administered to the human or other animal after irradiation in an amount that is effective for diminishing damage to the respiratory, gastrointestinal and the hematopoietic systems after sublethal irradiation or for increasing the survival rate after lethal irradiation. The gene therapy may also be effective when administered prior to or during exposure to radiation. Another dosing regimen would include multiple doses given both prior and/or following the exposure to radiation.

Those of skill in the art are well aware of how to apply adenoviral delivery to in vivo and ex vivo situations. For viral vectors, one generally will prepare a viral vector stock. Depending on the kind of virus and the titer attainable, one will deliver 1 to 10, 10 to 50, 100-1000, or up to 1×10⁴, 1×10⁵, 1×10⁶, 1×10⁷, 1×10⁸, 1×10⁹, 1×10¹⁰, 1×10¹¹, or 1×10¹² infectious particles to the patient in a pharmaceutically acceptable composition as discussed below.

Various routes are contemplated for various tumor types. Where discrete locations or tissues may be identified, a variety of direct, local and regional approaches may be taken. For example, an organ may be directly injected with the adenovirus. The adenovirus can be delivered by a catheter having access to the tissue. One may utilize the local vasculature to introduce the vector into the tissue or organ by injecting a supporting vein or artery. A more distal blood supply route also may be utilized. It may also be beneficial to treat the surrounding tissue, not just the affected tissue.

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

Appropriate salts and buffers can be used to render delivery vectors stable and allow for uptake by target cells. Aqueous compositions of the gene therapy can include an effective amount of the vectors, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the gene therapy, its use in therapeutic compositions is contemplated. Supplementary active ingredients also can be incorporated into the compositions.

An effective amount of the therapeutic agent is determined based on the intended goal, for example, lessening of cellular damage. The term “unit dose” refers to physically discrete units suitable for use in a subject, each unit containing a predetermined-quantity of the gene therapy composition calculated to produce the desired responses, discussed above, in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual. The engineered viruses of the may be administered directly into animals, or alternatively, administered to cells that are subsequently administered to animals.

The gene therapy may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The gene therapy compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, and 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, and parenteral vehicles such as sodium chloride or Ringer's dextrose. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters. When the route is topical, the form may be a cream, ointment, or salve.

In a further embodiment of the invention, an adenovirus or a nucleic acid encoding an adenovirus of the gene therapy may be delivered to cells using liposome or immunoliposome delivery. The adenovirus or nucleic acid encoding an adenovirus may be entrapped in a liposome or lipid formulation. Liposomes may be targeted to a cell by attaching antibodies to the liposome that bind specifically to a cell surface marker on the cell. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, Targeted Diagn Ther. 4:87-103 [1991]). Also contemplated is a nucleic acid construct complexed with Lipofectamine (Gibco BRL).

Additionally, radiation damage often occurs as a result of disease treatments, such as cancer treatments. This limits the ability of the physician to apply high doses of radiation that would otherwise be beneficial in treating the disease. The present embodiment provides a therapeutic that improves the efficacy of radiotherapy.

Traditional therapy to treat cancers may include removal of all or part of the affected organ, external beam irradiation, xenon arc, and argon laser photocoagulation, cryotherapy, immunotherapy, and chemotherapy. The choice of treatment is dependent on multiple factors, such as, 1) multifocal or unifocal disease, 2) site and size of the tumor, 3) metastasis of the disease, 4) age of the patient, or 5) histopathologic findings (The Genetic Basis of Human Cancer, 1998).

It is contemplated that adenoviral therapy could be used in conjunction with a radiotherapeutic intervention, as well as radiodiagnostic techniques. It also may prove effective to combine the therapeutic with other treatments that create similar cellular effects to that of radiation.

A “target” cell, which will be treated by the gene therapy described herein, may kill cells, inhibit cell growth, inhibit metastasis, inhibit angiogenesis or otherwise reverse or reduce a hyperproliferative phenotype of target cells. The gene therapy can be combined with another composition, both in effective amounts, can be used to kill or inhibit proliferation of the target cell while protecting the normal cells. This process may involve contacting the cells with the expression construct and the agent(s) or factor(s) at the same or different times. This may be achieved by contacting the cell with a single composition or pharmacological formulation that includes both agents, or by contacting the cell with two distinct compositions or formulations, wherein one composition includes the gene therapy and the other includes the second agent.

It also is conceivable that more than one administration of either the gene therapy and/or the second agent will be desired. Various combinations may be employed, where the gene therapy is “A” and the other agent is “B”, for example: A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Other combinations are contemplated to achieve normal cell survival and decrease or limit normal cell damage resulting from the administration of another therapeutic or treatment.

In another embodiment, the combination therapy includes the gene therapy, for the treatment of radiation damage, which is described in detail above, in combination with a chemotherapeutic, which is administered post-radiation treatment. This combination allows more aggressive treatment of the disease causing cellular damage with either no or limited amounts of damage to the normal cells of the subject.

Agents or factors suitable for use in a combined therapy are any anti-angiogenic agent and/or any chemical compound or treatment method with anticancer activity; therefore, the term “anticancer agent” that is used throughout this application refers to an agent with anticancer activity. These compounds or methods include alkylating agents, topoisomerase I inhibitors, topoisomerase II inhibitors, RNA/DNA antimetabolites, DNA antimetabolites, antimitotic agents, as well as DNA damaging agents, which induce DNA damage when applied to a cell.

The gene therapy may also be combined with immunosuppression. The immunosuppression may be performed as described in WO 96/12406, which is incorporated herein by reference. Examples of immunosuppressive agents include cyclosporine, FK506, cyclophosphamide, and methotrexate.

Alternatively, the gene therapy administration may precede or follow the second agent or treatment by intervals ranging from minutes to weeks. In embodiments where the second agent and gene therapy are applied separately to the cell, one would generally ensure that a significant period of time did not expire between the times of each delivery, such that the second agent and gene therapy would still be able to exert an advantageously combined effect on the cell. In such instances, it is contemplated that one would contact the cell with both modalities within about 12-24 hours of each other and, possibly, within about 6-12 hours of each other, with a delay time of only about 12 hours being most common. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

The above discussion provides a factual basis for the use of the compositions of the above embodiments. The method used with and the utility of the embodiments can be shown by the following non-limiting examples and accompanying figures.

### EXAMPLES

### General Methods

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds.) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### Example 1

### Materials and Methods

### Adenoviral Vectors

The following adenoviral vectors were used in the study:

(1). Ad5CMVECSOD: a replication-deficient recombinant adenovirus carrying the human extracellular superoxide dismutase (ECSOD) gene under the control of a cytomegalovirus (CMV) promoter (Chu et al. Circ Res. 92:461-8 [2003]); and

(2). Ad5CMVntlacZ: a replication-deficient recombinant adenovirus carrying the nuclear-targeted β-galactosidase reporter gene ntlacZ under the control of a CMV promoter (Chu et al. Circ Res. 92:461-8 [2003])

Both adenoviral vectors were purchased from the University of Iowa Gene Transfer Vector Core (Iowa City, Iowa).

Isolation and Ex Vivo Expansion of Mouse Mesenchymal Stem Cells (mMSCs)

mMSCs were isolated as previously described (Deng et al., Am J Physiol Cell Physiol 285:C1322-9 [2003]; Sun et al., Stem Cells. 21:527-35 [2003]; Peister et al. Blood 103:1662-8 [2004]; Bivalacqua et al., Am J Physiol Heart Circ Physiol. 292:H1278-90 [2007]; Abdel-Mageed et al., Blood 113: 1201-3 [2009]). Six-week-old female BALB/c mice (The Jackson Laboratory, Bar Harbor, Me.) were euthanized with CO₂ and femurs and tibias were removed.

Both ends of the bones were cut and bone marrow was flushed out using a 18-gauge needle and culture medium for mMSCs [MEM-α (Atlanta Biologicals, Norcross, Ga.); 20% fetal bovine serum (FBS, GIBCO Invitrogen Corp., Carlsbad, Calif.); 100 units/ml penicillin, 100 μg/ml streptomycin, and 250 ng/ml amphotericin B (Atlanta Biologicals); and 2 mM L-glutamine (GIBCO Invitrogen Corp)]. The bone marrow cells were filtered through a cell strainer with 70-μm nylon mesh (BD Bioscience, Bedford, Mass.), and the cells from each mouse were plated in a T75 flask (Falcon, Fisher Scientific, Pittsburgh, Pa.). The cells were incubated at 37° C. with 5% humidified CO₂, and mMSCs were isolated by their adherence to tissue culture plastic. Fresh culture medium was added and replaced every 2-3 days. The adherent mMSCs were grown to 90% confluence, harvested with 0.25% trypsin/1 mM EDTA for 2 minutes at 37° C., and diluted 1:3 for ex vivo expansion.

Adenoviral Transduction of mMSCs to Secrete Biologically Active ECSOD

mMSCs were transduced with adenoviral vectors as previously described (Deng et al., Stem Cells 22: 1279-91 [2004]; Baber et al., Am J Physiol Heart Circ Physiol. 292: H1120-8 [2007]; Abdel-Mageed et al., Blood 113: 1201-3 [2009]). Briefly, mMSCs were plated at a density of 10,000 cells/cm² in 6-well plates or T75 flasks (Falcon, Fisher Scientific) and incubated overnight. The cells were counted and then exposed to fresh culture medium containing Ad5CMVECSOD at 0 or 2000 multiplicities of infection (M01) for 48 hours. MOI is defined as pfu/cell. Virus-containing culture medium was discarded, cells were washed 3 times with PBS, and fresh culture medium was added. Cells were counted, cultured for 48 hours, and culture supernatant was collected. The culture supernatant was then assayed for the secretion of biologically active ECSOD by Ad5CMVECSOD-transduced mMSCs using a SOD activity assay kit (Cayman Chemical Company, Ann Arbor, Mich.). Data were expressed as mean±SEM (n=3).

Adenoviral Transduction of mMSCs to Express β-galactosidase

mMSCs were transduced with adenoviral vectors as previously described (Deng et al., Am J Physiol Cell Physiol. 285:C1322-9 [2003]; Deng et al., Life Sci. 78: 1830-8 [2006]; Abdel-Mageed et al., Blood 113: 1201-3 [2009]). Briefly, mMSCs were plated at a density of 10,000 cells/cm² in 6-well plates or T75 flasks (Falcon, Fisher Scientific) and incubated overnight. The cells were counted and then exposed to fresh culture medium containing Ad5CMVntlacZ at 0 or 2000 MOI for 48 hours. To conduct X-gal cytochemistry for β-galactosidase activity, the transduced mMSCs were washed with PBS, fixed for 5 minutes in fixing solution (2% formaldehyde, 0.2% glutaraldehyde, Sigma, St. Louis, Mo.), washed twice with PBS, and incubated in staining solution (1 mg/ml X-gal, 5 mM K ferricyanide, 5 mM K ferrocyanide, and 2 mM MgCl₂, Sigma) at 37° C. in the dark overnight. Cells were washed with PBS and the expression of transgene ntlacZ in mMSCs was evaluated by light microscopy scoring of blue cells expressing the nuclear-targeted β-galactosidase activity (Deng et al. Stem Cells 22:1279-91 [2004]; Abdel-Mageed et al., Blood 113: 1201-3 [2009]).

In Vitro Differentiation of mMSCs into Osteoblasts or Adipocytes

In vitro differentiation of mMSCs into osteoblast or adipocyte lineages was conducted as previously described (Deng et al., Am J Physiol Cell Physiol 285:C1322-9 [2003]; Deng et al. Stem Cells. 22:1279-91 [2004]). Cells in 6-well plates or T75 flasks were treated with culture medium for mMSCs plus either osteogenic supplement (1×10⁻⁵ mM dexamethasone, 0.2 mM ascorbic acid, and 10 mM β-glycerol phosphate, Sigma) or adipogenic supplement (0.5 μM hydrocortisone, 500 μM isobutylmethylxanthine, and 60 μM indomethacin, Sigma). The differentiation medium was changed every 3 days until day 21. To assess mineral deposition, cells were washed with PBS, fixed with cold methanol (−20° C.) for 10 minutes, washed with dH₂O twice, stained with 2% Alizarin red S (pH 4.1, Sigma) for 15 minutes, washed with dH₂O five times, and checked under an inverted phase contrast microscope. To assess lipid droplet formation, cells were washed with PBS, fixed with 10% formalin (Sigma) for 1 hour, washed with dH₂O twice, stained with a freshly prepared Oil red O solution for 15 minutes, washed with dH₂O, and checked under an inverted phase contrast microscope. The Oil red O solution was prepared by mixing three parts of an Oil red O stock solution (0.5%, prepared in isopropanol, Sigma) with two parts of dH₂O and filtering through a 0.45 μm pore size filter.

Isolation and Culture of Human Mesenchymal Stem Cells (hMSCs)

hMSCs were isolated as previously described (Deng et al. Biochem Biophys Res Commun 282:148-52 [2001]; Mageed et al. Transplantation 83:1019-26 [2007]). About 10 ml bone marrow aspirate were taken from the iliac crest of normal donors. The bone marrow aspirate was diluted 1:1 with Hanks' Balanced Salt Solution (HBSS, GIBCO Invitrogen Corp.) and layered over 15 ml Ficoll (Fico/Lite LymphoH, Atlanta Biologicals). After centrifugation at 400×g for 30 minutes, the mononuclear cell layer was recovered from the gradient interface and washed with HBSS. The cells were suspended in 10 ml culture medium for hMSCs [MEM-α (Atlanta Biologicals); 20% fetal bovine serum (FBS, GIBCO Invitrogen Corp.); 100 units/ml penicillin and 100 μg/ml streptomycin (Atlanta Biologicals); and 2 mM L-glutamine (GIBCO Invitrogen Corp.)]. All of the cells were plated in one T75 flask (Falcon, Fisher Scientific) and incubated at 37° C. with 5% humidified CO₂. Three days later, the culture medium was discarded to remove non-adherent cells and hMSCs were isolated by their adherence to tissue culture plastic. Fresh culture medium was added and replaced every 2-3 days. The adherent hMSCs were grown to 70-90% confluency over about 14 days. The cells were harvested with 0.25% Trypsin/1 mM EDTA for 5 minutes at 37° C. and diluted 1:3 for ex vivo expansion.

Adenoviral Transduction of hMSCs to Secrete Biologically Active ECSOD

hMSCs were transduced with adenoviral vectors as previously described (Deng et al., Stem Cells 22: 1279-91 [2004]; Baber et al., Am J Physiol Heart Circ Physiol. 292: H1120-8 [2007]). hMSCs were plated at a density of 10,000 cells/cm² in 6-well plates or T75 flasks and incubated overnight. The cells were counted and then exposed to fresh culture medium containing Ad5CMVECSOD at 0 or 2000 MOI for 48 hours. Virus-containing culture medium was discarded, cells were washed 3 times with PBS, and fresh culture medium was added. Cells were then counted, cultured for 48 hours, and culture supernatant was collected. The culture supernatant was then assayed for the secretion of biologically active ECSOD by Ad5CMVECSOD-transduced hMSCs using a SOD activity assay kit (Cayman Chemical Company). Data were expressed as mean±SEM (n=3).

Adenoviral Transduction of hMSCs to Express β-Galactosidase

hMSCs were transduced with adenoviral vectors as previously described (Deng et al., Am J Physiol Cell Physiol 285:C1322-9 [2003]. Deng et al., Life Sci. 78: 1830-8 [2006]). hMSCs were plated at a density of 10,000 cells/cm² in 6-well plates or T75 flasks (Falcon, Fisher Scientific) and incubated overnight. The cells were counted and then exposed to fresh culture medium containing Ad5CMVntlacZ at 0 or 2000 MOI for 48 hours. To conduct X-gal cytochemistry for β-galactosidase activity, the transduced hMSCs were washed with PBS, fixed for 5 minutes in fixing solution (2% formaldehyde, 0.2% glutaraldehyde, Sigma), washed twice with PBS, and incubated in staining solution (1 mg/ml X-gal, 5 mM K ferricyanide, 5 mM K ferrocyanide, and 2 mM MgCl₂, Sigma) at 37° C. in the dark overnight. Cells were washed with PBS and the expression of transgene ntlacZ in hMSCs was evaluated by light microscopy scoring of blue cells expressing the nuclear-targeted β-galactosidase activity (Deng et al. Stem Cells 22:1279-91 [2004]).

In Vitro Differentiation of hMSCs into Osteoblasts or Adipocytes

In vitro differentiation of hMSCs into osteoblast or adipocyte lineages was conducted as previously described (Deng et al. Biochem Biophys Res Commun. 282:148-52 [2001]; Deng et al., Am J Physiol Cell Physiol. 285:C1322-9 [2003]. Deng et al., Stem Cells. 22:1279-91 [2004]). Cells in 6-well plates or T75 flasks (Falcon, Fisher Scientific) were treated with culture medium for hMSCs plus either osteogenic supplement (1×10⁻⁵ mM dexamethasone, 0.2 mM ascorbic acid, and 10 mM β-glycerol phosphate, Sigma) or adipogenic supplement (0.5 μM hydrocortisone, 500 μM isobutylmethylxanthine, and 60 μM indomethacin, Sigma). The differentiation medium was changed every 3 days until day 21. To assess mineral deposition, cells were washed with PBS, fixed with cold methanol (−20° C.) for 10 minutes, washed with dH₂O twice, stained with 2% Alizarin red S (pH 4.1, Sigma) for 15 minutes, washed with dH₂O five times, and checked under an inverted phase contrast microscope. To assess lipid droplet formation, cells were washed with PBS, fixed with 10% formalin (Sigma) for 1 hour, washed with dH₂O twice, stained with a freshly prepared Oil red 0 solution for 15 minutes, washed with dH₂O, and checked under an inverted phase contrast microscope. The Oil red O solution was prepared by mixing three parts of an Oil red O stock solution (0.5%, prepared in isopropanol, Sigma) with two parts of dH₂O and filtering through a 0.45 μm pore size filter.

Intravenous Administration of Adenovirus Carrying ECSOD or ntlacZ Gene into Irradiated Mice Through Tail Vein Injection

Five-week-old female BALB/c mice were given 9 Gy total body γ irradiation from a ¹³⁷Cs source (Gammacell 1000; MDS Nordion, Ottawa, ON) at a dose rate of 1.28 Gy/min. Twenty-four hours later, these animals received a tail vein injection of 200 μl phosphate buffered saline (PBS), 1×10⁸ pfu Ad5CMVntlacZ in 200 μl PBS, or 1×10⁸ pfuAd5CMVECSOD in 200 μl PBS. All in vivo experiments were performed on mice in accordance with institutional and NIH guidelines for the care and use of laboratory animals. The mice were then monitored 35 days for mouse body weight and survival. Each value represented mean±SEM (n=8).

### Statistical Analysis

Data were expressed as mean±SEM. A Kaplan-Meier survival curve was used for analyzing mouse survival data analysis.

### Results

### Radiation Damage in Mice is Dose-Dependent.

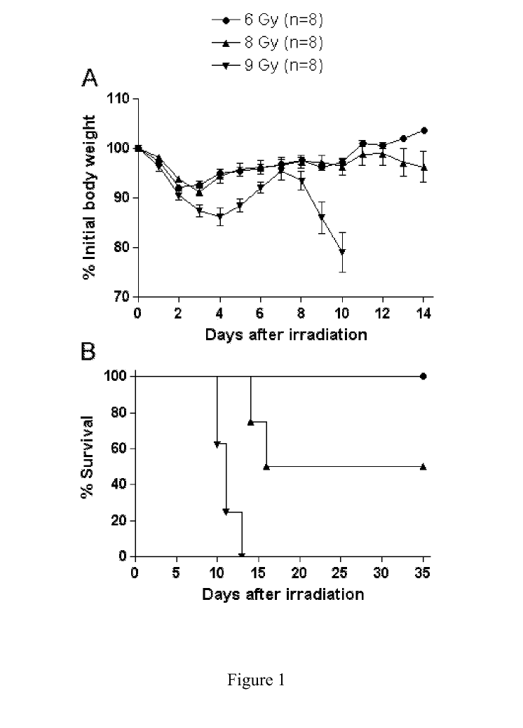

To determine whether radiation damage is dose-dependent, 5-week-old female BALB/c mice were given 6, 8, or 9 Gy total body y irradiation from a ¹³⁷Cs source at a dose rate of 1.28 Gy/min. Mouse body weight and survival were then monitored every day for 35 days. As shown in FIGS. 1A and 1B, the effect of irradiation on mouse body weight loss and survival is dose-dependent.

FIGS. 1A and 1B show that radiation damage is dose dependent. Five-week-old female BALB/c mice were given 6, 8, or 9 Gy total body y irradiation from a ¹³⁷Cs source. Mouse body weight and survival were then monitored every day for 35 days. FIG. 1A is a graph depicting the effect of irradiation dose on mouse body weight loss. FIG. 1B is a Kaplan-Meier survival curve depicting the effect of irradiation dose on mouse survival. Each value represented mean±SEM (n=8). The Kaplan-Meier survival curve was used for data analysis and statistical significance was determined between groups using logrank test and one-way analysis of variance (ANOVA) followed by post hoc analysis with Tukey test. P<0.05 was considered statistically significant. The difference between the 3 groups was statistically significant by logrank test (P<0.0001) and ANOVA test (P<0.0001). Further, P<0.001 for ▾vs, P<0.05 for ▾vs▴, and P<0.05 for ▴vs by Tukey test

### Example 2

### Improvement of Survival of Irradiated Mice by Intravenous Administration of Adenovirus Carrying ECSOD Gene

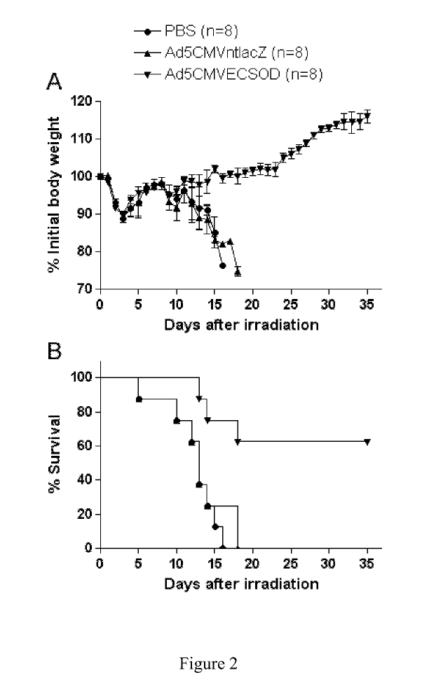

To determine whether intravenous administration of adenovirus carrying ECSOD gene has a therapeutic effect on radiation damage, 5-week-old female BALB/c mice were given 9 Gy total body y irradiation from a ¹³⁷Cs source. Twenty-four hours later, the animals were given a tail vein injection of PBS, 1×10⁸ pfu Ad5CMVntlacZ, or 1×10⁸ pfu Ad5CMVECSOD. Mouse body weight and survival were then monitored daily for 35 days. As shown in FIG. 2A, mice in Ad5CMVECSOD treatment group started to gain weight at about 12 days after irradiation whereas mice in PBS or Ad5CMVntlacZ treatment group started to lose weight at about 12 days after irradiation. As shown in FIG. 2B, approximately 63% of animals in Ad5CMVECSOD treatment group survived for over 35 days whereas no animals in PBS or Ad5CMVntlacZ treatment group survived more than 35 days. Therefore, intravenous treatment with adenovirus carrying ECSOD gene after whole-body radiation exposure improves survival.

More specifically, FIGS. 2A and 2B show that in vivo extracellular superoxide dismutase (ECSOD) gene therapy has a radioprotective effect in irradiated mice. Five-week-old female BALB/c mice were given 9 Gy total body y irradiation from a ¹³⁷Cs source. About 24 hours later, the animals were given a tail vein injection of phosphate buffered saline (PBS), Ad5CMVntlacZ, or Ad5CMVECSOD. Mouse body weight and survival were monitored every day for 35 days. FIG. 2A is a graph depicting the effect of intravenous adenoviral-mediated gene transfer of ECSOD on mouse body weight loss in irradiated mice. FIG. 2B is a Kaplan-Meier survival curve demonstrating that intravenous treatment with adenovirus carrying ECSOD gene improves survival of irradiated mice. Each value represented mean±SEM (n=8). The Kaplan-Meier survival curve was used for data analysis and statistical significance was determined between groups using logrank test and one-way analysis of variance (ANOVA) followed by post hoc analysis with Tukey test. P<0.05 was considered statistically significant. The difference between the 3 groups was determined to be statistically significant by logrank test (P=0.0098) and ANOVA (P=0.0004). Further, P<0.01 for ▾vs, P<0.01 for ▾vs▴, and P>0.05 for ▴vs by Tukey test.

### Example 3

### Secretion of Biologically Active ECSOD by mMSCs Genetically Modified with ECSOD

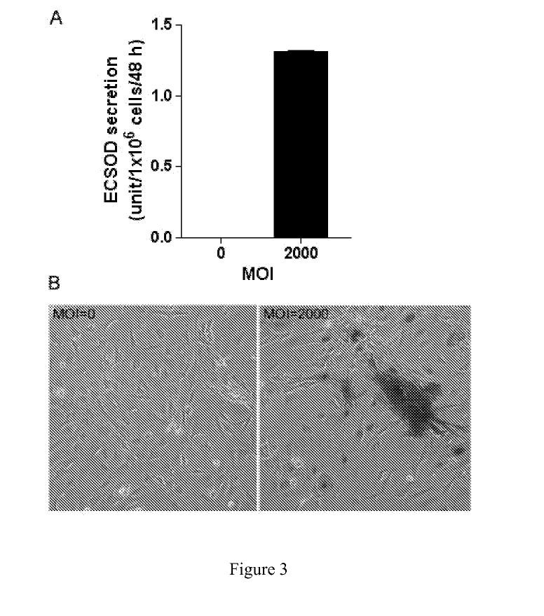

To ascertain whether Ad5CMVECSOD can infect mMSCs and whether mMSCs genetically modified with ECSOD, also known as ECSOD gene-modified mMSCs or Ad5CMVECSOD-transduced mMSCs, can produce and secrete functional ECSOD, mMSCs were transduced with Ad5CMVECSOD at MOI 0 or 2000 for 48 hours. The cells were washed with PBS and further incubated for 48 hours. The culture supernatant was then collected and analyzed for SOD activity. FIG. 3A shows the secretion of biologically active ECSOD by Ad5CMVECSOD-transduced mMSCs. The efficacy of adenoviral-mediated gene transfer into mMSCs was further examined using the reporter gene ntlacZ. To this end, mMSCs were transduced with Ad5CMVntlacZ at MOI 0 or 2000. After 48 hours, the expression of nuclear-targeted β-galactosidase in Ad5CMVntlacZ-transduced mMSCs was assessed by X-gal staining. FIG. 3B shows the expression of nuclear-targeted β-galactosidase by Ad5CMVntlacZ-transduced mMSCs. Therefore, adenoviral transduction of mMSCs is effective and ECSOD gene-modified mMSCs produce and secrete biologically active ECSOD.

More specifically, FIGS. 3A and 3B show the efficacy of adenoviral gene transfer in mouse mesenchymal stem cells (mMSCs). FIG. 3A is a graph depicting the secretion of biologically active extracellular superoxide dismutase (ECSOD) by Ad5CMVECSOD-transduced mMSCs. mMSCs were transduced with Ad5CMVECSOD at MOI 0 or 2000 for 48 hours, the virus-containing culture medium was removed and cells were washed 3 times with PBS and further incubated in fresh culture medium for 48 hours. The culture supernatant was collected and analyzed for superoxide dismutase (SOD) activity using a SOD activity assay kit (Cayman Chemical Company, Ann Arbor, Mich.). Data were expressed as mean±SEM (n=3). FIG. 3B is a set of images depicting the expression of nuclear targeted β-galactosidase by Ad5CMVntlacZ-transduced mMSCs. mMSCs were transduced with Ad5CMVntlacZ at MOI 0 or 2000 for 48 hours. The cells were then X-gal stained for β-galactosidase activity and the blue nuclear-targeted β-galactosidase positive Ad5CMVntlacZ-transduced mMSCs were identified. Original magnification: ×40.

### Example 4

### Secretion of Biologically Active ECSOD by hMSCs Genetically Modified with ECSOD

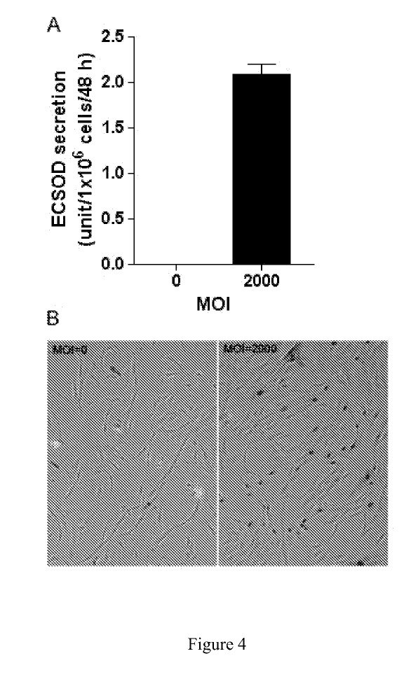

To ascertain whether Ad5CMVECSOD can infect hMSCs and whether hMSCs genetically modified with ECSOD, also known as ECSOD gene-modified hMSCs or Ad5CMVECSOD-transduced hMSCs, can produce and secrete functional ECSOD, hMSCs were transduced with Ad5CMVECSOD at MOI 0 or 2000 for 48 hours. The cells were washed with PBS and further incubated for 48 hours. The culture supernatant was then collected and analyzed for SOD activity. FIG. 4A shows the secretion of biologically active ECSOD by Ad5CMVECSOD-transduced hMSCs. The efficacy of adenoviral-mediated gene transfer into hMSCs was further examined using the reporter gene ntlacZ. To this end, hMSCs were transduced with Ad5CMVntlacZ at MOI 0 or 2000. After 48 hours, the expression of nuclear-targeted β-galactosidase in Ad5CMVntlacZ-transduced hMSCs was assessed by X-gal staining. FIG. 4B shows the expression of nuclear-targeted β-galactosidase by Ad5CMVntlacZ-transduced hMSCs. Therefore, adenoviral transduction of hMSCs is effective and ECSOD gene-modified hMSCs produce and secrete biologically active ECSOD.

More specifically, FIGS. 4A and 4B show the efficacy of adenoviral gene transfer in human mesenchymal stem cells (hMSCs) also known as human marrow stromal cells. FIG. 4A is a graph depicting the secretion of biologically active extracellular superoxide dismutase (ECSOD) by Ad5CMVECSOD-transduced hMSCs. hMSCs were transduced with Ad5CMVECSOD at MOI 0 or 2000 for 48 hours, the virus-containing culture medium was removed and cells were washed 3 times with PBS and further incubated in fresh culture medium for 48 hours. The culture supernatant was collected and analyzed for superoxide dismutase (SOD) activity using a SOD activity assay kit (Cayman Chemical Company, Ann Arbor, Mich.). Data were expressed as mean±SEM (n=3). FIG. 4B is a photomicrograph depicting the expression of nuclear targeted β-galactosidase by Ad5CMVntlacZ-transduced hMSCs. hMSCs were transduced with Ad5CMVntlacZ at MOI 0 or 2000 for 48 hours. The cells were then X-gal stained for β-galactosidase activity and the blue nuclear-targeted β-galactosidase positive Ad5CMVntlacZ-transduced hMSCs were identified. Original magnification:×40.

The above description is that of the current embodiment of the invention. Various alterations and changes can be made without departing from the spirit and broader aspects of the invention as defined in the appended claims, which are to be interpreted in accordance with the principles of patent law including the doctrine of equivalents.

The embodiments of the invention in which an exclusive property or privilege is claimed are defined as follows:

1. A method of at least one of treating and preventing radiation damage by:
administering to a patient in need of treatment at least one therapeutically effective amount of a vector carrying an extracellular superoxide dismutase gene to produce and secrete exogenous extracellular superoxide dismutase.

2. The method of claim 1, wherein said administering step includes administering in a manner selected from the group consisting of intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and combinations thereof.

3. The method of claim 1, wherein said administering step includes administering the vector at a location selected from the group consisting of systemically, at the site of injury, at an adjacent site to the site of injury, and at a site remote from the site of injury, wherein the vehicle migrates to the site of injury after administration.

4. The method of claim 1, wherein said administering step includes administering to a patient in need of treatment multiple therapeutically effective amounts of the vector.

5. The method of claim 1, wherein said administering step includes administering the vector in combination with another therapeutic.

6. A therapeutic for treating and/or preventing radiation related damage, said therapeutic comprising:
a vector carrying an extracellular superoxide dismutase gene to produce and secrete exogenous extracellular superoxide dismutase.

7. The therapeutic of claim 6, wherein said vector is selected from the group consisting of adenovirus vectors, retrovirus vectors, adeno-associated virus vectors, herpes simplex virus vectors, SV 40 vectors, polyoma virus vectors, papilloma virus vectors, picarnovirus vectors, vaccinia virus vectors, a helper-dependent adenovirus, a gutless adenovirus, and a plasmid.

8. The therapeutic of claim 6, wherein said extracellular superoxide dismutase gene is a gene selected from the group consisting of human extracellular superoxide dismutase gene and murine extracellular superoxide dismutase gene.

9. The therapeutic of claim 6 for use in treating radiation damage or radiation-like damage.

10. The therapeutic of claim 9, wherein said radiation damage is damage selected from the group consisting of cell injury, tissue damage, organ dysfunction, acute radiation syndrome, radiation-induced lifespan shortening, cataractogenesis, and carcinogenesis.

11. The therapeutic of claim 6 for use in preventing radiation damage or radiation-like damage.

12. The therapeutic of claim 11, wherein said vector is administered prior to exposure to radiation to prevent damage to normal tissue.

13. The therapeutic of claim 11, wherein said vector is combined with another unrelated therapy.

14. A combination therapy for treating or preventing radiation and radiation-like damage, said therapy comprising:
a vector carrying an extracellular superoxide dismutase gene to produce and secrete exogenous extracellular superoxide dismutase; and
an unrelated therapeutic.

15. The therapy of claim 14, wherein said vector is selected from the group consisting of adenovirus vectors, retrovirus vectors, adeno-associated virus vectors, herpes simplex virus vectors, SV 40 vectors, polyoma virus vectors, papilloma virus vectors, picarnovirus vectors, vaccinia virus vectors, a helper-dependent adenovirus, a gutless adenovirus, and plasmid.

16. The therapy of claim 14, wherein said extracellular superoxide dismutase gene is a gene selected from the group consisting of human extracellular superoxide dismutase gene and murine extracellular superoxide dismutase gene.

17. The therapy of claim 14, wherein said unrelated therapeutic is selected from the group consisting of a radiotherapeutic and chemotherapeutic.
